(19) United States
(12) Patent Application Publication
(10) Pub. No.: US 2021/0340476 A1
(43) Pub. Date: Nov. 4, 2021

(54) CONTAINER WITH EMBEDDED STRUCTURE

(71) Applicant: ORIGEM BIOTECHNOLOGY INC., Taichung City (TW)

(72) Inventors: Hao Chen CHANG, Taichung City (TW); Hung Jui CHEN, Taichung City (TW)

(21) Appl. No.: 17/286,452

(22) PCT Filed: Feb. 22, 2019
(86) PCT No.: PCT/CN2019/075810

§ 371 Date: Apr. 17, 2021

(51) Int. Cl. C12M 1/32; C12M 1/00; C12M 1/12

(52) U.S. Cl. CPC C12M 23/12; C12M 25/06; C12M 23/26

(57) ABSTRACT

A container with an embedded structure mainly includes: a first plate with a first plate body, a first end surface located on one side of the first plate body; a second plate with a second plate body, a second end surface opposite to the first end surface and located on one side of the second plate body, a flexible protrusion is annularly protruded on the second end surface and abuts against the first plate to form an accommodating space between the first end surface, the second end surface, and the protrusion for a fluid to flow in the accommodating space, and the accommodating space has an accommodating height between the first end surface and the second end surface; and a shape of a portion of a free end of the protrusion abutting the first end surface is deformed.

### BACKGROUND OF THE INVENTION

### Field of Invention

The invention relates to containers, and more particularly relates to a container with an embedded structure.

### Related Art

According to the development of biomedical technology, biotechnology has been widely used in the fields of agriculture, food industry and medicine, and biotechnology includes the technologies of genetic engineering, cell culture and tissue culture. Among the technologies, cell culture is one of the basic implementation technologies for biomedical related research work. A common cell culture technology places cells in a multi-well microplate and grows them in a static culture method under sterile, proper temperature and nutrient conditions. About a week later, each of the cells will grow into a colony to achieve an object of proliferation.

In addition, in order to ensure that the cells are cultured in an environment free from external interference, the multi-well microplate is usually covered with an outer cover. However, due to the conventional combination between the multi-well microplate and the outer cover is mainly achieved by means of screw connection between a screw and a screw hole, the use of such method shows that the above-mentioned conventional structure is inconvenient for closing or opening the outer cover. Furthermore, if a user does not securely lock the outer cover, an inside of the multi-well microplate will be connected to the outside, besides the possibility of leakage, it may also have an adverse effect on the growth of cells, and therefore experimental errors are increased.

### SUMMARY OF THE INVENTION

Therefore, a main object of the invention is to provide a container with an embedded structure, which has a simplified structure, in addition to being convenient to assemble and disassemble, an accommodating space is formed between two plates, while ensuring the accommodating space is kept in a liquid-tight state to avoid leakage.

Another object of the invention is to provide a container with an embedded structure capable of being used for cell culture and applied to biomedical cell detection.

Therefore, in order to achieve the above-mentioned objects, the invention provides a container with an embedded structure comprising: a first plate with a first plate body, a first end surface located on one side of the first plate body; a second plate with a second plate body, a second end surface opposite to the first end surface and located on one side of the second plate body, a flexible protrusion being annularly protruded on the second end surface and abutting against the first plate to form an accommodating space between the first end surface, the second end surface, and the protrusion for a fluid to flow in the accommodating space, and the accommodating space having an accommodating height between the first end surface and the second end surface; and a shape of a portion of a free end of the protrusion abutting the first end surface being deformed.

Wherein the first plate further includes a concave portion complementarily embedded with the protrusion, and recessed with a predetermined depth from the first end surface toward an inner direction of the first plate body.

In one embodiment of the invention, the protrusion further includes a first convex body inserted in the concave portion, a free end of the first convex body abuts against a bottom surface of the concave portion, a height of the first convex body protruding outside of the second end surface is greater than a depth of the concave portion, and a portion of the first convex body exposed outside of the concave portion constitutes the accommodating height. With the aforementioned structure, in addition to facilitating simple and rapid assembly or separation between the first and second plates, structural corresponding design of the protrusion and the concave portion is capable of forming the accommodating space between the first and second plates.

In one embodiment of the invention, the protrusion further includes a first convex body inserted in the concave portion, and a second convex body disposed on one side of the first convex body, a height of the first convex body protruding outside of the second end surface is greater than a height of the second convex body protruding outside of the second end surface, and a stopping distance between the free end of the first convex body and a free end of the second convex body is smaller than the depth of the concave portion, so that the free end of the second convex body abuts on the first end surface, and a height of the second convex body constitutes the accommodating height. With the aforementioned structure, in addition to facilitating simple and rapid assembly or separation between the first and second plates, structural corresponding design of the protrusion and the concave portion is capable of forming the accommodating space between the first and second plates.

In one embodiment of the invention, the protrusion further includes a first convex body inserted in the concave portion, a height of the convex body protruding from the second end surface is not greater than a depth of the concave portion; the first plate further includes a groove located on the first end surface and surrounded by the concave portion, a predetermined groove depth is recessed from the first end surface toward the inner direction of the first plate body, and the groove depth of the groove constitutes the accommodating height. With the aforementioned structure, in addition to facilitating simple and rapid assembly or separation between the first and second plates, structural corresponding design of the groove is capable of forming the accommodating space between the first and second plates.

In one embodiment of the invention, the first plate further includes a groove located on the first end surface and surrounded by the concave portion, a predetermined groove depth is recessed from the first end surface toward the inner direction of the first plate body; the protrusion further includes a columned first convex body inserted in the concave portion, and a shouldered second convex body with a predetermined thickness disposed on one side of the first convex body, a height of the first convex body protruding outside of the second end surface is greater than a height of the second convex body protruding outside of the second end surface, and a stopping distance between the free end of the first convex body and the free end of the second convex body is smaller than the depth of the concave portion, so that the free end of the second convex body abuts on the first end surface, and disposes between the free end of the first convex body and the first end surface, and is used to abut on the bottom surface of the concave portion, and a sum of the groove depth of the groove and the height of the shouldered second convex body constitutes the accommodating height. With the aforementioned structure, in addition to facilitating simple and rapid assembly or separation between the first and second plates, structural corresponding design of the protrusion, the concave portion and the groove is capable of forming the accommodating space between the first and second plates.

Further, in each of the above embodiments, an axial sectional width of the protrusion along its own annular center is not less than an axial sectional width of the concave portion along its own annular center, flexible physical properties of the protrusion allow the protrusion to squeeze into the concave portion with a slight deformation to achieve a tight embedding state, and to be capable of increasing a contact area between the protrusion and the concave portion, thereby enhancing a bonding strength.

In another embodiment, further including a third plate; the third plate has a third end surface, the second plate has a fourth end surface located on another side of the second plate body and opposite to the second end surface, and the third end surface of the third plate is attached with the fourth end surface of the second plate, so that the second plate is sandwiched and pressed tightly between the first plate and the third plate. Thereby, a portion of the protrusion abutting on the first end surface is deformed due to being pressed to ensure its liquid tightness and avoid the problem of leakage.

Wherein the axial sectional width of the protrusion along its own annular center is not greater than the axial sectional width of the concave portion along its own annular center.

In order to achieve the above disclosed objects, the invention also provides a container with an embedded structure comprising: a first plate with a first plate body, a first end surface located on one side of the first plate body; and a flexible second plate with a second plate body, a second end surface opposite to the first end surface and located on one side of the second plate body, a sleeve portion annularly protruded on the second end surface to sleeve on the first plate to achieve a tightly connected state between the first plate and the sleeve portion in order to form an accommodating space between the first end surface, the second end surface, and the sleeve portion for a fluid to flow in the accommodating space, and the accommodating space has an accommodating height between the first end surface and the second end surface.

Wherein the second plate is made of flexible material.

Wherein the first plate further includes a protruding portion protruding on the first end surface, an outer diameter of the protruding portion is greater than an inner diameter of the sleeve portion, when an inner side surface of the sleeve portion is sleeved on a peripheral side of the protruding portion, the second plate is deformed to change the inner diameter of the sleeve portion. Thereby, facilitating simple and rapid assembly or separation between the first and second plates.

In one embodiment of the invention, a height of the protruding portion protruding from the first end surface is smaller than a height of the sleeve portion protruding from the second end surface, and an accommodating space is formed between the first and second plates.

In one embodiment of the invention, the first plate further includes a groove located on the protruding portion, a predetermined groove depth is recessed from the first end surface toward the inner direction of the first plate body, and a groove depth of the groove constitutes the accommodating height.

In one embodiment of the invention, the sleeve portion further includes a first sleeve body sleeved on the peripheral side of the protruding portion, and a second sleeve body provided on one side of the first sleeve body, a height of the first sleeve body protruding outside of the second end surface is greater than a height of the second sleeve body protruding outside of the second end surface, and a stopping distance between a free end of the first sleeve body and a free end of the second sleeve body is smaller than the depth of the concave portion, so that the free end of the second sleeve body abuts on the protruding portion, and a height of the second sleeve body constitutes the accommodating height.

In one embodiment of the invention, the first plate further includes a groove located on the protruding portion, a predetermined groove depth is recessed from the first end surface toward the inner direction of the first plate body; the sleeve portion further includes a first sleeve body sleeved on the peripheral side of the protruding portion, and a second sleeve body provided on one side of the first sleeve body, a height of the first sleeve body protruding outside of the second end surface is greater than a height of the second sleeve body protruding outside of the second end surface, and a stopping distance between the free end of the first sleeve body and the free end of the second sleeve body is smaller than the depth of the concave portion, so that the free end of the second sleeve body abuts on the protruding portion, and a sum of the groove depth of the groove and the height of the second sleeve body constitutes the accommodating height.

### BRIEF DESCRIPTION OF THE DRAWINGS

FIG. 1 is an assembled perspective view of a first embodiment of the invention;

FIG. 2 is a cross-sectional view of FIG. 1 of the first embodiment of the invention;

FIG. 3 is an exploded perspective view of the first embodiment of the invention;

FIG. 4 is a cross-sectional view of FIG. 3 of the first embodiment of the invention;

FIG. 5 is an exploded perspective view of a second embodiment of the invention;

FIG. 6 is a cross-sectional view of FIG. 5 of the second embodiment of the invention;

FIG. 7 is a cross-sectional view of exploded state of each of components of the second embodiment of the invention;

FIG. 8 is a cross-sectional view of assembled state of each of components of a third embodiment of the invention;

FIG. 9 is a cross-sectional view of exploded state of each of components of the third embodiment of the invention;

FIG. 10 is a cross-sectional view of assembled state of each of components of a fourth embodiment of the invention;

FIG. 11 is a cross-sectional view of exploded state of each of components of the fourth embodiment of the invention;

FIG. 12 is a cross-sectional view of assembled state of each of components of a fifth embodiment of the invention;

FIG. 13 is a cross-sectional view of exploded state of each of components of the fifth embodiment of the invention;

FIG. 14 is an assembled perspective view of a sixth embodiment of the invention;

FIG. 15 is a cross-sectional view of FIG. 14 of the sixth embodiment of the invention;

FIG. 16 is an exploded perspective view of the sixth embodiment of the invention;

FIG. 17 is a cross-sectional view of FIG. 16 of the sixth embodiment of the invention;

FIG. 18 is a cross-sectional view of assembled state of each of components of a seventh embodiment of the invention;

FIG. 19 is a cross-sectional view of exploded state of each of components of the seventh embodiment of the invention;

FIG. 20 is a cross-sectional view of assembled state of each of components of an eighth embodiment of the invention;

FIG. 21 is a cross-sectional view of exploded state of each of components of the eighth embodiment of the invention;

FIG. 22 is a cross-sectional view of assembled state of each of components of a ninth embodiment of the invention;

FIG. 23 is a cross-sectional view of exploded state of each of components of the ninth embodiment of the invention;

FIG. 24 is an assembled perspective view of a tenth embodiment of the invention;

FIG. 25 is a cross-sectional view of FIG. 24 of the tenth embodiment of the invention;

FIG. 26 is an exploded perspective view of the tenth embodiment of the invention;

FIG. 27 is a cross-sectional view of FIG. 26 of the tenth embodiment of the invention;

FIG. 28 is a cross-sectional view of assembled state of each of components of an eleventh embodiment of the invention;

FIG. 29 is a cross-sectional view of exploded state of each of components of the eleventh embodiment of the invention;

FIG. 30 is a cross-sectional view of assembled state of each of components of a twelfth embodiment of the invention;

FIG. 31 is a cross-sectional view of exploded state of each of components of the twelfth embodiment of the invention;

FIG. 32 is a cross-sectional view of assembled state of each of components of a thirteenth embodiment of the invention; and

FIG. 33 is a cross-sectional view of exploded state of each of components of the thirteenth embodiment of the invention.

### DETAILED DESCRIPTION OF THE INVENTION

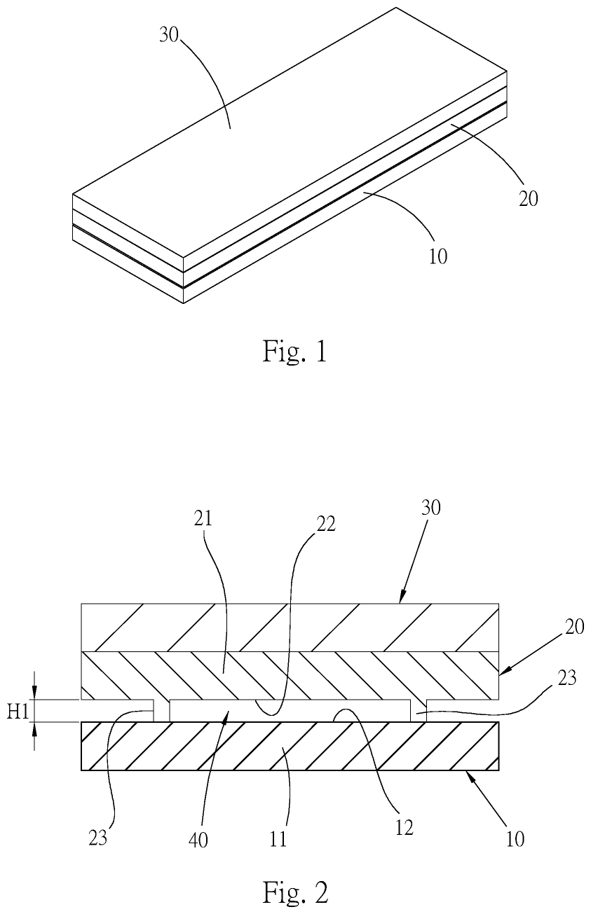

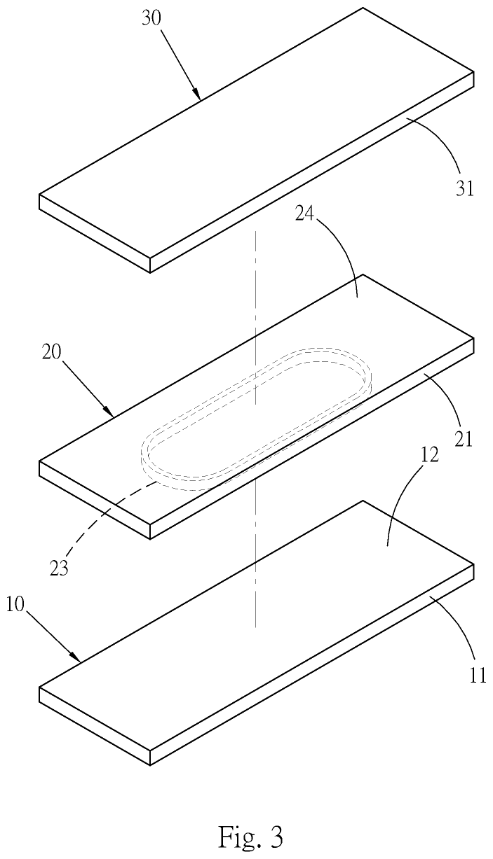

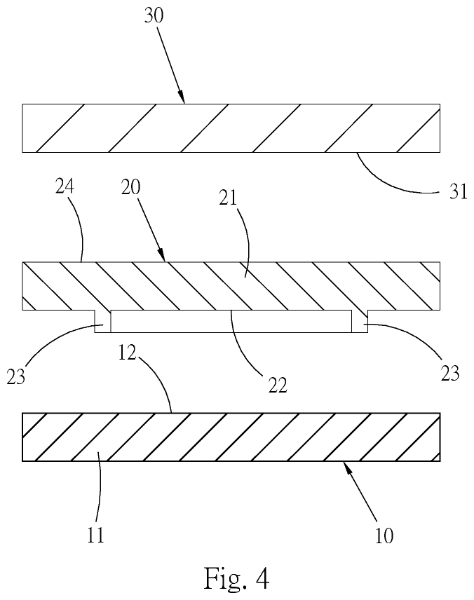

First of all, please refer to FIGS. 1 to 4, a container with an embedded structure provided in a first embodiment of the invention mainly includes a first plate 10, a second plate 20 and a third plate 30, each of the plates is generally in a shape of an elongated sheet, the second plate 20 is interposed between the first plate 10 and the third plate 30, the first plate 10 is connected with the second plate 20 by embedding means, and an accommodating space 40 is formed between the first plate 10 and the second plate 20.

The first plate 10 is made of hard plastic material, and the first plate 10 has a first plate body 11 and a first end surface 12 located on one side of the first plate body 11. Wherein the first plate 10 is made by injection molding, and then further micro-engraving is used to cut out required lines and contour. Of course, the first plate 10 can also be made by precise one-piece molding without the need for secondary engraving.

The second plate 20 is a flexible body made of silicone rubber material, the second plate 20 has a second plate body 21, a second end surface 22 opposite to the first end surface 12 and located on one side of the second plate body 21, a flexible protrusion 23 annularly protruded on the second end surface 22, and a fourth end surface 24 located on another side of the second plate body 21 and opposite to the second end surface 22.

The third plate 30 is made of hard plastic material, and the third plate 30 has a third end surface 31 that is attached to the fourth end surface 24 oppositely. The first plate 10 and the third plate 30 can be connected through a butting structure, so that the second plate 20 is sandwiched and forced to be located between the first plate 10 and the third plate 30, and the second plate 20 is subjected to a pressing force. Wherein the butting structure can be but not limited to snap components.

With composition of the above-mentioned components, the container with the embedded structure is capable of making combination of the first plate 10 and the second plate 20 in a tightly connected state. As shown in FIG. 2, under an effect of the aforementioned pressing force, due to compressible, deformable and flexible elasticity of silicone rubber material, the protrusion 23 abuts against a free end portion of the first end surface 12, and deforms in shape, thereby causing the protrusion 23 to be closely attached to the first end surface 12 to achieve an air-tight state to avoid fluid leakage. Furthermore, with the protrusion 23 existing between the first plate 10 and the second plate 20, the first end surface 12 and the second end surface 22 are separated from each other, thereby forming an appropriate accommodating height H1 between the first end surface 12 and the second end surface 22, and forming the accommodating space 40 between the first end surface 12, the second end surface 22 and the protrusion 23 as a flow space for a fluid to flow between the first end surface 12 and the second end surface 22.

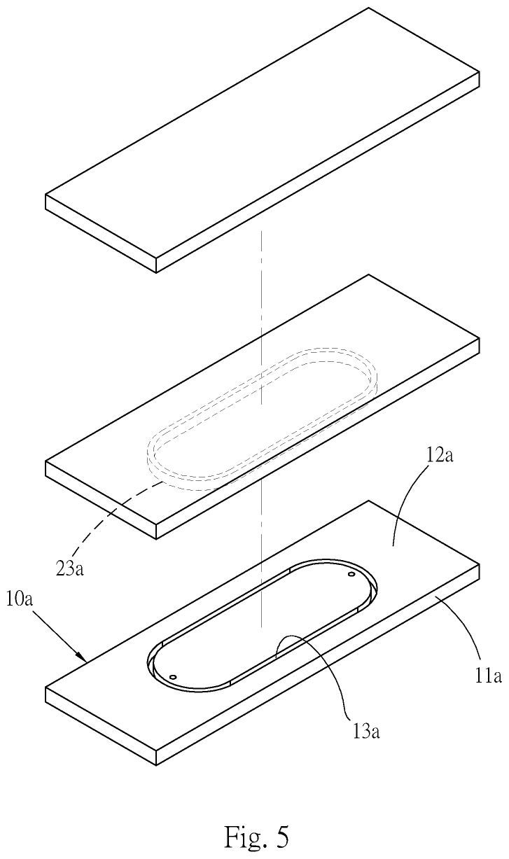

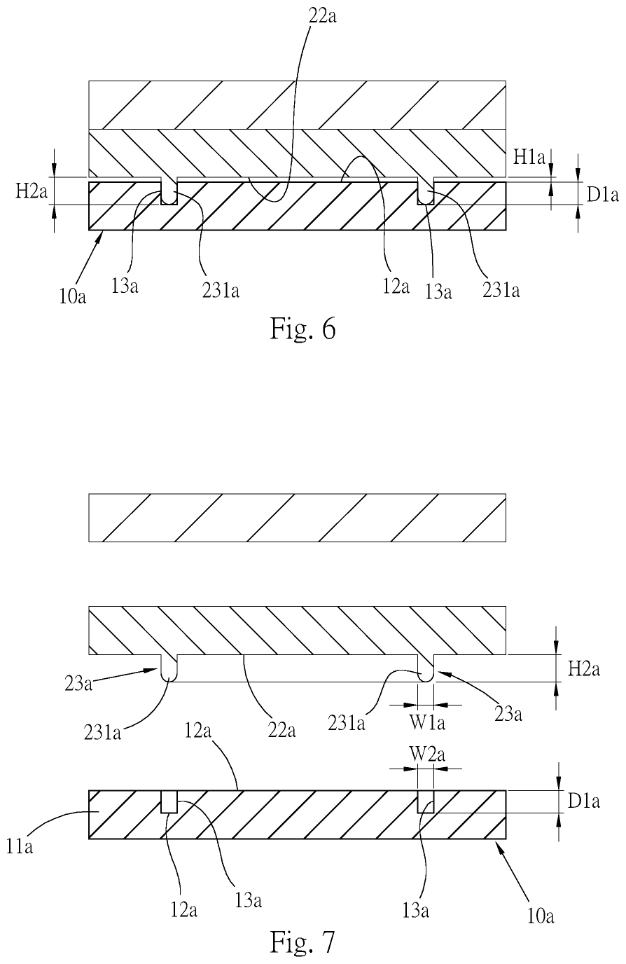

Please refer to FIG. 5 to FIG. 7 for a second embodiment of the invention with main differences different from the first embodiment lying in the following.

In the second embodiment, the first plate 10a further includes a concave portion 13a that is complementarily embedded with a protrusion 23a, and a predetermined depth D1a is recessed from a first end surface 12a toward an inner direction of a first plate body 11a. Furthermore, the protrusion 23a further includes a first convex body 231a inserted into the concave portion 13a. During a process of embedding the protrusion 23a into the concave portion 13a, a free end of the first convex body 231a reaches into the concave portion 13a until the free end abuts on a bottom surface of the concave portion 13a, so that a portion of the first convex body 231a is inserted into the concave portion 13a, thereby providing a quick and convenient positioning method. Since a height H2a of the first convex body 231a protruding from a second end surface 22a is greater than the depth D1a of the concave portion 13a, the first convex body 231a will not be completely submerged in the concave portion 13a, and a portion of the first convex body 231a exposed outside of the concave portion 13a constitutes an accommodating height H1a. At the same time, in order to make the first convex body 231a and the concave portion 13a fit tightly to achieve an air-tight state and avoid fluid leakage, an axial sectional width W1a of the protrusion 23a along its own annular center is further made equal to an axial sectional width W2a of the concave portion 13a along its own annular center, and flexible physical properties of the protrusion 23a allow the first convex body 231a to be slightly deformed to squeeze into the concave portion 13a to achieve a tightly embedded state. In other embodiments, the axial sectional width W1a of the protrusion 23a along its own annular center can be smaller than the axial sectional width W2a of the concave portion 13a along its own annular center, and only the free end of the first convex body 231a is used to press against the bottom surface of the concave portion 13a to achieve an air-tight effect.

Wherein a cross-sectional shape of the free end of the first convex body 231a can be, but is not limited to, any one of a group consisting of arc, tapered, round, and ellipse.

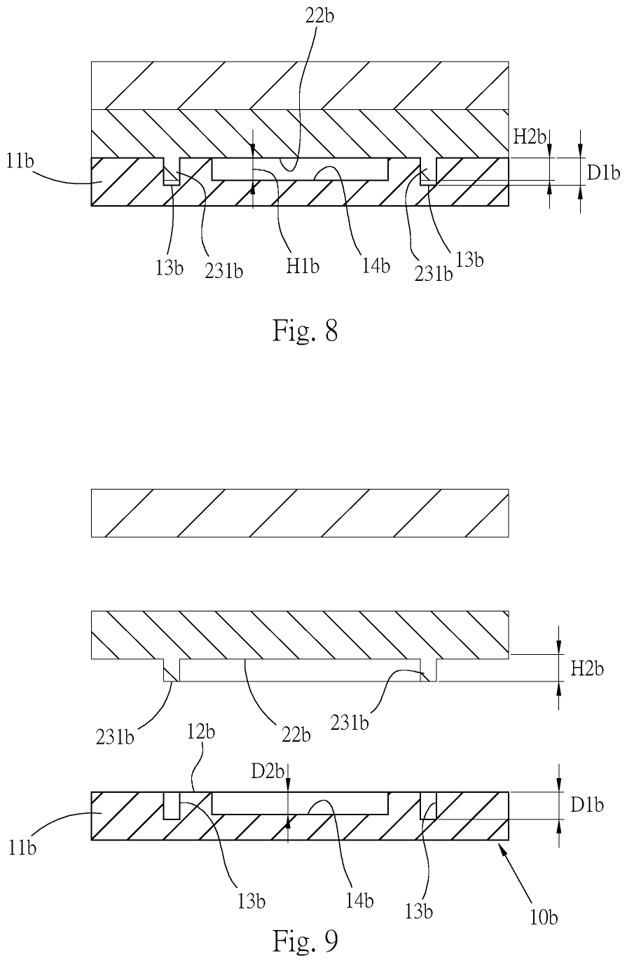

Please refer to FIG. 8 and FIG. 9 for a third embodiment of the invention with main differences different from the aforementioned second embodiment lying in a first plate 10b further including a groove 14b located on a first end surface 12b and surrounded by a concave portion 13b, and a predetermined groove depth D2b is recessed from the first end surface 12b toward an inner direction of a first plate body 11b. Furthermore, since a height H2b of a first convex body 231b protruding from a second end surface 22b is equal to a depth D1b of the concave portion 13b, the first convex body 231b is exactly accommodated in the concave portion 13b, and the second end surface 22b is correspondingly attached to the first end surface 12b of a periphery of the groove 14b, so that the groove depth D2b of the groove 14b constitutes an accommodating height H1b.

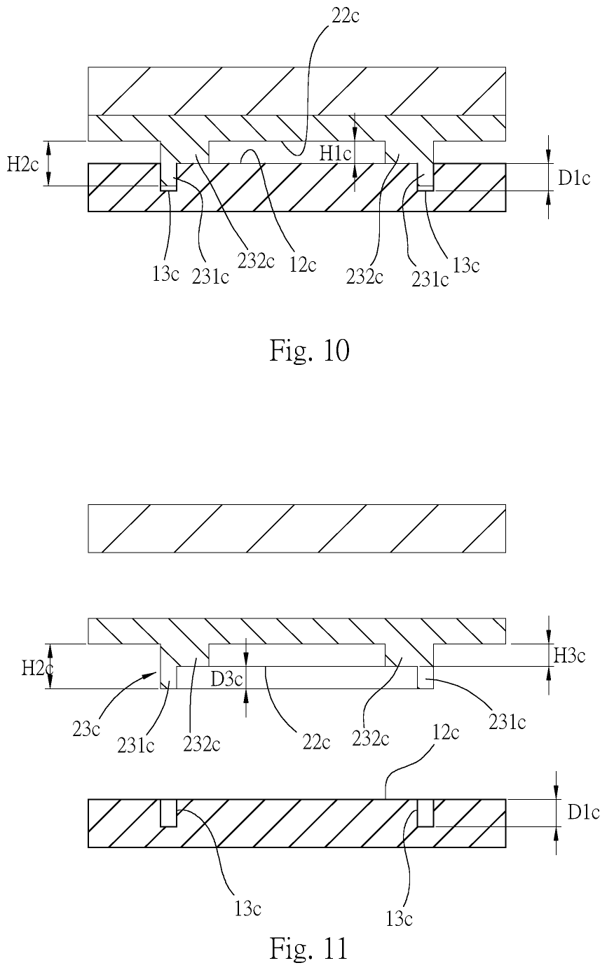

Please refer to FIG. 10 and FIG. 11 for a fourth embodiment of the invention with main differences different from the aforementioned second embodiment lying in a protrusion 23c further including a second convex body 232c disposed on one side of a first convex body 231c, a height H2c of the first convex body 231c protruding from a second end surface 22c is greater than a height H3c of the second convex body 232c protruding from the second end surface 22c, and a stopping distance D3c between a free end of the first convex body 231c and a free end of the second convex body 232c is smaller than a depth D1c of a concave portion 13c, so that the free end of the second convex body 232c abuts on a first end surface 12c, and a height H3c of the second convex body 232c constitutes an accommodating height Elk. In a process of embedding the protrusion 23c into the concave portion 13c, in order to provide a quick and convenient positioning method between the protrusion 23c and the concave portion 13c, the free end of the first convex body 231c first reaches into the concave portion 13c, since the depth D1c of the concave portion 13c is greater than the stopping distance D3c, the free end of the second convex body 232c abuts and stops on the first end surface 12c, and then under an effect of the aforementioned pressing force, flexible physical properties of the protrusion 23c allow the second convex body 232c to be slightly deformed to achieve an airtight embedding state.

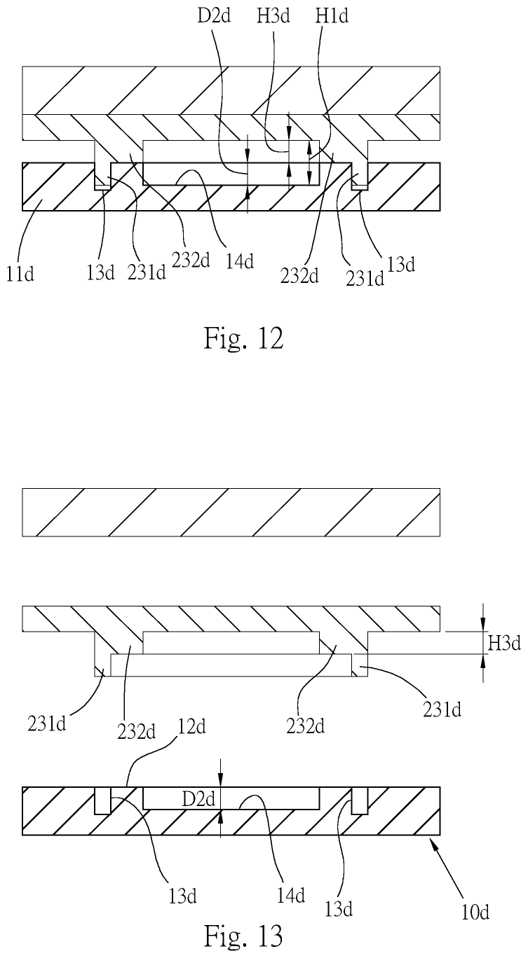

Please refer to FIG. 12 and FIG. 13 for a fifth embodiment of the invention with main differences different from the aforementioned fourth embodiment lying in a first plate 10d further including a groove 14d located on a first end surface 12d and surrounded by a concave portion 13d, and a predetermined groove depth D2d is recessed from the first end surface 12d toward an inner direction of a first plate body 11d. Furthermore, since a first convex body 231d and a second convex body 232d are of unequal structural design, a free end of the second convex body 232d abuts on the first end surface 12d, and a sum of the groove depth D2d of the groove 14d and a height H3d of the second convex body 232d constitutes an accommodating height H1d.

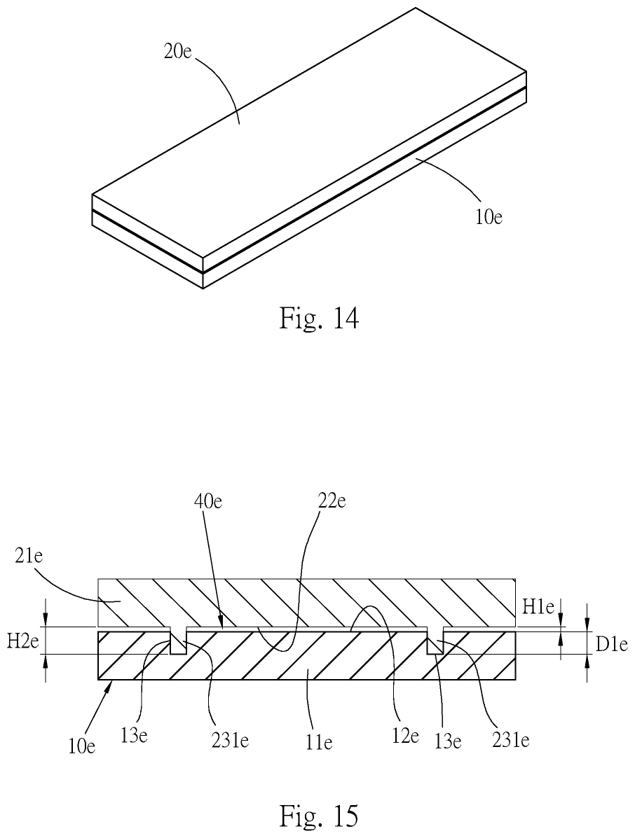

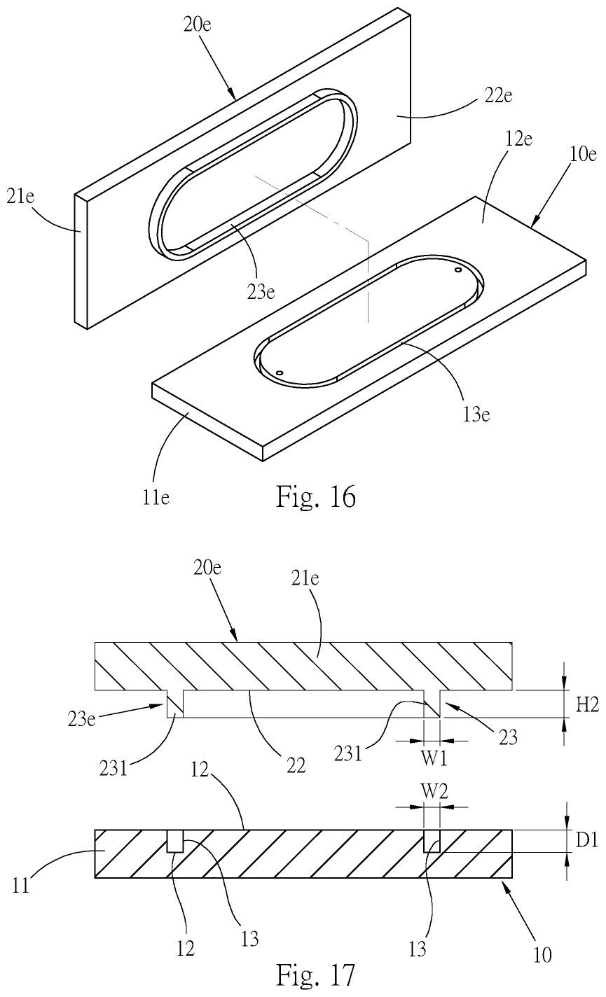

Please refer to FIGS. 14 to 17, the container with the embedded structure provided in a sixth embodiment of the invention mainly includes a first plate 10e and a second plate 20e, each of the plates is generally in a shape of an elongated sheet, the first plate 10e and the second plate 20e are connected by means of embedding, and at the same time, an accommodating space 40e is formed between the first plate 10e and the second plate 20e.

The first plate 10e is made of hard plastic material, and the first plate 10e has a first plate body 11e, a first end surface 12e located on one side of the first plate body 11e, and a concave portion 13e with a predetermined depth D1e recessed from the first end surface 12e toward an inner direction of the first plate body 11e. Wherein the first plate 10e is made by injection molding, and then further micro-engraving is used to cut out required lines and contour. Of course, the first plate 10e can also be made by precise one-piece molding without the need for secondary engraving.

The second plate 20e is a flexible body made of silicone rubber material, and the second plate 20e has a second plate body 21e, a second end surface 22e opposite to the first end surface 12e and located on one side of the second plate body 21e, and a flexible protrusion 23e annularly protruded on the second end surface 22e for complementarily embedding with the concave portion 13e, and the protrusion 23e is capable of reliably sealing a gap between the first plate 10e and the second plate 20e to completely prevent leakage of material in the accommodating space 40e.

In a specific implementing method, the protrusion 23e further includes a first convex body 231e inserted into the concave portion 13e. During a process of embedding the protrusion 23e into the concave portion 13e, a free end of the first convex body 231e reaches into the concave portion 13e until the free end abuts on a bottom surface of the concave portion 13e, so that a portion of the first convex body 231e is inserted into the concave portion 13e, thereby providing a quick and convenient positioning method. Since a height H2e of the first convex body 231e protruding from the second end surface 22e is greater than the depth D1e of the concave portion 13e, the first convex body 231e will not be completely submerged in the concave portion 13e, and a portion of the first convex body 231e exposed outside of the concave portion 13e constitutes an accommodating height H1e. At the same time, in order to make the first convex body 231e and the concave portion 13e fit tightly to achieve an air-tight state and avoid fluid leakage, an axial sectional width W1e of the protrusion 23e along its own annular center is further made not less than an axial sectional width W2e of the concave portion 13e along its own annular center, and flexible physical properties of the protrusion 23e allow the first convex body 231e to be slightly deformed to squeeze into the concave portion 13e to achieve a tightly embedded state.

Wherein a cross-sectional shape of the free end of the first convex body 231e can be, but is not limited to, any one of a group consisting of arc, tapered, round, and ellipse.

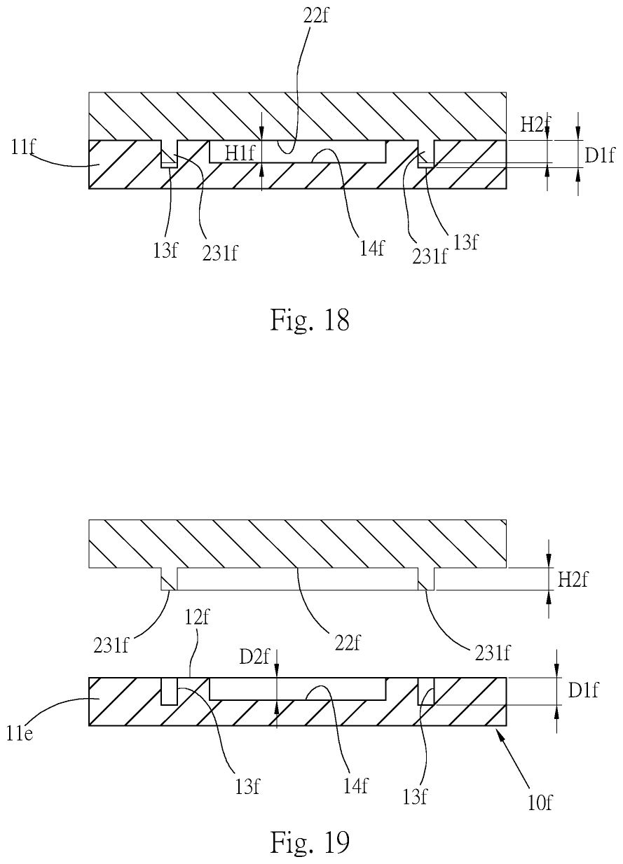

Please refer to FIG. 18 and FIG. 19 for a seventh embodiment of the invention with main differences different from the aforementioned sixth embodiment lying in a first plate 10f further including a groove 14f located on a first end surface 12f and surrounded by a concave portion 13f, and a predetermined groove depth D2f is recessed from the first end surface 12f toward an inner direction of a first plate body 11f. Furthermore, since a height H2f of a first convex body 231f protruding from a second end surface 22f is equal to a depth D1f of the concave portion 13f, the first convex body 231f is exactly accommodated in the concave portion 13f, and the second end surface 22f is correspondingly attached to the first end surface 12f of a periphery of the groove 14f, so that the groove depth D2f of the groove 14f constitutes an accommodating height H1f.

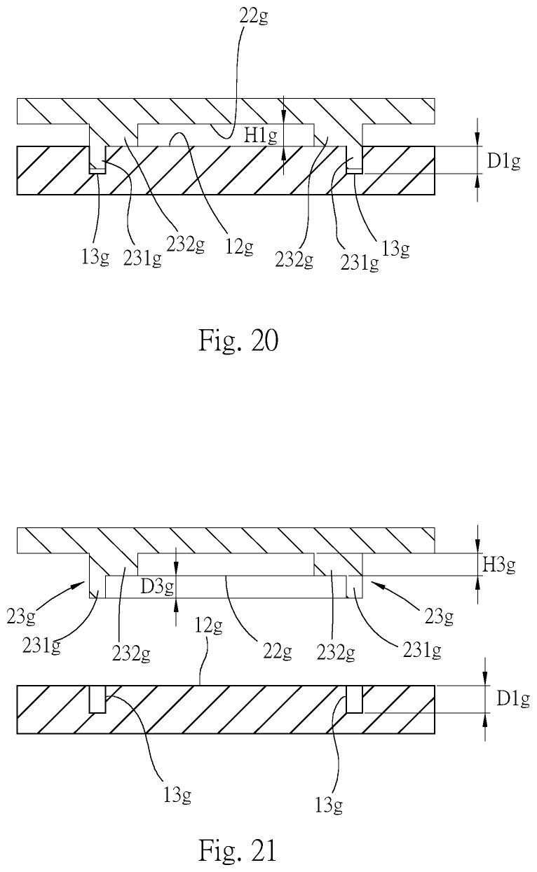

Please refer to FIG. 20 and FIG. 21 for an eighth embodiment of the invention with main differences different from the aforementioned sixth embodiment lying in a protrusion 23g further including a second convex body 232g disposed on one side of a first convex body 231g, a height H2g of the first convex body 231g protruding from a second end surface 22g is greater than a height H3g of the second convex body 232g protruding from the second end surface 22g, and a stopping distance D3g between a free end of the first convex body 231g and a free end of the second convex body 232g is smaller than a depth D1g of a concave portion 13g, so that the free end of the second convex body 232g abuts on a first end surface 12g, and a height H3g of the second convex body 232g constitutes an accommodating height H1g. In a process of embedding the protrusion 23g into the concave portion 13g, in order to provide a quick and convenient positioning method between the protrusion 23g and the concave portion 13g, the free end of the first convex body 231g first reaches into the concave portion 13g, since the depth D1g of the concave portion 13g is greater than the stopping distance D3g, the free end of the second convex body 232g abuts and stops on the first end surface 12g, and then under an effect of the aforementioned pressing force, flexible physical properties of the protrusion 23g allow the second convex body 232g to be slightly deformed to achieve an airtight embedding state.

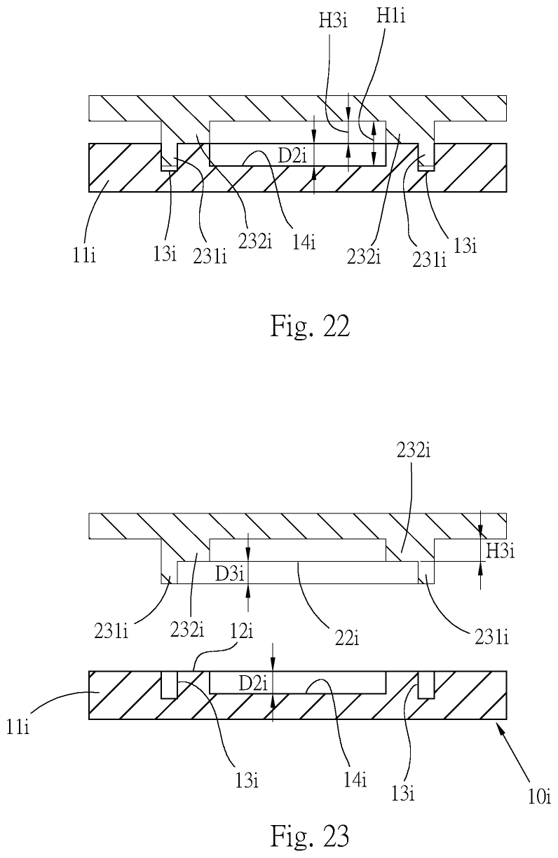

Please refer to FIG. 22 and FIG. 23 for a ninth embodiment of the invention with main differences different from the aforementioned eighth embodiment lying in a first plate 10i further including a groove 14i located on a first end surface 12i and surrounded by a concave portion 13i, and a predetermined groove depth D2i is recessed from the first end surface 12i toward an inner direction of a first plate body 11i. Furthermore, since a first convex body 231i and a second convex body 232i are of unequal structural design, a free end of the second convex body 232i abuts on the first end surface 12i, and a sum of the groove depth D2i of the groove 14i and a height H3i of the second convex body 232i constitutes an accommodating height H1i.

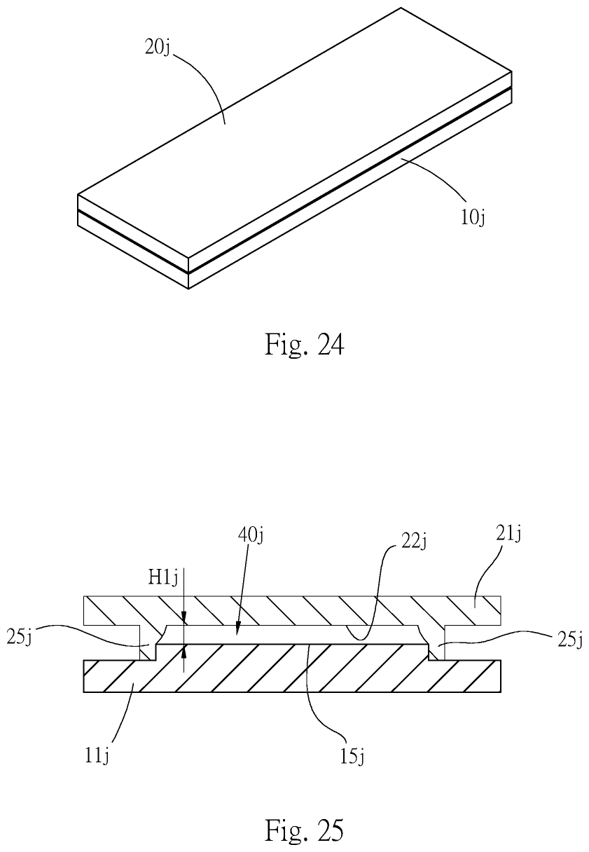

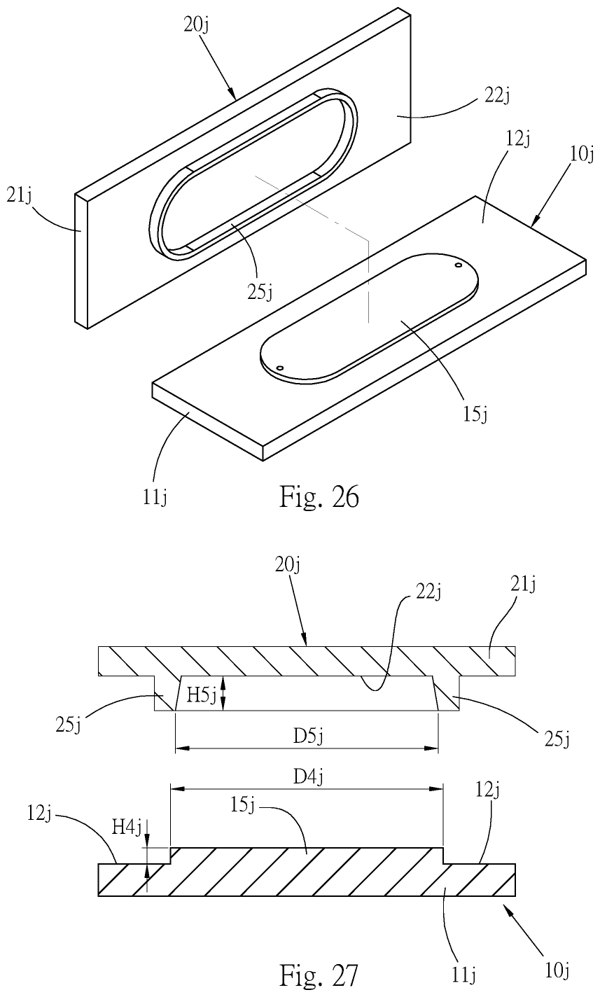

Please refer to FIGS. 24 to 27, the container with the embedded structure provided in a tenth embodiment of the invention mainly includes a first plate 10j and a second plate 20j, each of the plates is generally in a shape of an elongated sheet, the first plate 10j and the second plate 20j are connected by means of sleeving, and at the same time, an accommodating space 40j is formed between the first plate 10j and the second plate 20j.

The first plate 10j is made of hard plastic material, and the first plate 10j has a first plate body 11j, a first end surface 12j located on one side of the first plate body 11j, and a protruding portion 15j protruded on the first end surface 12j. Wherein the first plate 10j is made by injection molding, and then further micro-engraving is used to cut out required lines and contour. Of course, the first plate 10j can also be made by precise one-piece molding without the need for secondary engraving.

The second plate 20j is a flexible body made of silicone rubber material, and the second plate 20j has a second plate body 21j, a second end surface 22j opposite to the first end surface 12j and located on one side of the second plate body 21j, and a sleeve portion 25j annularly protruded on the second end surface 22j to sleeve on the protruding portion 15j of the first plate 10j to achieve a tightly connected state between the first plate 10j and the sleeve portion 25j in order to reliably seal a gap between the first plate 10j and the second plate 20j to completely prevent leakage of material in the accommodating space 40j.

In a specific implementing method, in order to provide a quick and convenient positioning method between the protruding portion 15j and the sleeve portion 25j, an outer diameter D4j of the protruding portion 15j is greater than an inner diameter D5j of the sleeve portion 25j. During an assembly process, due to compressible, deformable and flexible elasticity of silicone rubber material, through an external acting force, the second plate 20j is deformed to change the inner diameter D5j of the sleeve portion 25j, thereby an inner side surface of the sleeve portion 25j is sleeved on a peripheral side of the protruding portion 15j, and finally the protruding portion 15j and the sleeve portion 25j are tightly fitted to achieve an airtight state to avoid fluid leakage.

Furthermore, a height H4j of the protruding portion 15j protruding from the first end surface 12j is smaller than a height H5j of the sleeve portion 25j protruding from the second end surface 22j, so that the accommodating space 40j is formed between the first end surface 12j, the second end surface 22j and the sleeve portion 25j for a fluid to flow in the accommodating space 40j, and the accommodating space 40j has an accommodating height H1j between the first end surface 12j and the second end surface 22j, that is, a difference between the height H4j of the protruding portion 15j and the height H5j of the sleeve portion 25j is the accommodating height H1j.

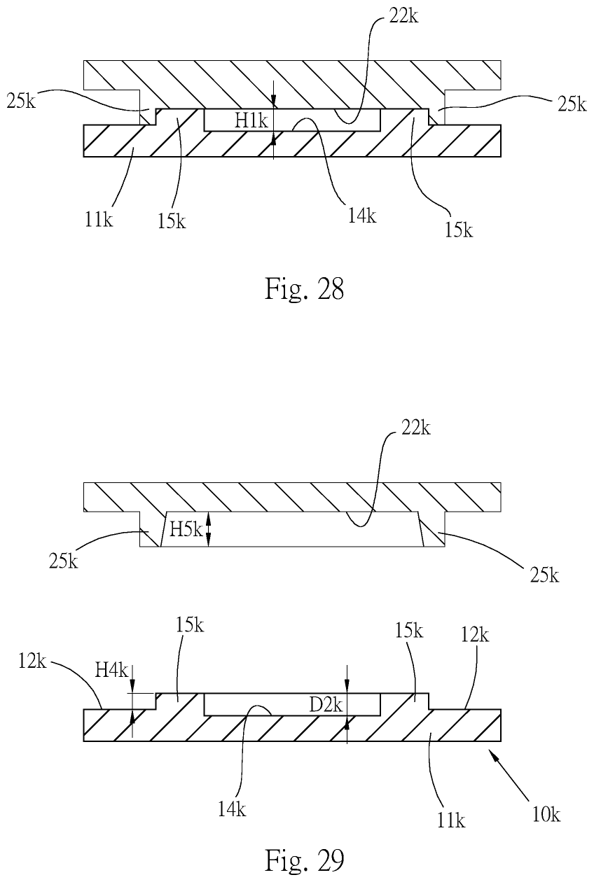

Please refer to FIG. 28 and FIG. 29 for an eleventh embodiment of the invention with main differences different from the aforementioned tenth embodiment lying in a first plate 10k further including a groove 14k located on a protruding portion 15k, and a predetermined groove depth D2k is recessed from a first end surface 12k toward an inner direction of a first plate body 11k. Furthermore, since a height H4k of the protruding portion 15k protruding from the first end surface 12k is not greater than a height H5k of a sleeve portion 25k protruding from a second end surface 22k; after the protruding portion 15k and the sleeve portion 25k are combined, the second end surface 22k is correspondingly attached on the first end surface 12k of a periphery of the groove 14k, and the groove depth D2k of the groove 14k constitutes an accommodating height H1k.

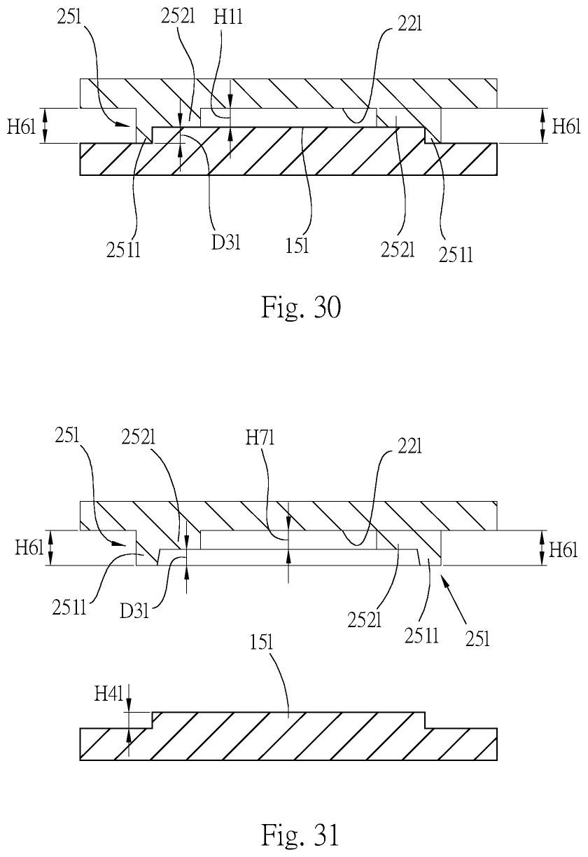

Please refer to FIG. 30 and FIG. 31 for a twelfth embodiment of the invention with main differences different from the aforementioned tenth embodiment lying in a sleeve portion 25l further including a first sleeve body 251l sleeved on a peripheral side of a protruding portion 15l, and a second sleeve body 252l provided on one side of the first sleeve body 251l, a height H61 of the first sleeve body 251l protruding outside of a second end surface 22l is greater than a height H7l of the second sleeve body 252l protruding outside of the second end surface 22l, and a stopping distance D31 between a free end of the first sleeve body 251l and a free end of the second sleeve body 252l is smaller than a height H41 of the protruding portion 15l, so that the free end of the second sleeve body 252l abuts on the protruding portion 15l, and a height H7l of the second sleeve body 252l constitutes an accommodating height H11.

In a process of sleeving the sleeve portion 25l on the protruding portion 15l, in order to provide a quick and convenient positioning method between the sleeve portion 25l and the protruding portion 15l, an inner wall surface of a portion of the first sleeve body 251l protruding and exposing from the second sleeve body 252l first sleeves against the peripheral side of the protruding portion 15l, and flexible physical properties of a protrusion 231 allow the free end of the second sleeve body 252l to be slightly deformed to abut and stop on the first end surface 12c to achieve a double airtight embedding state.

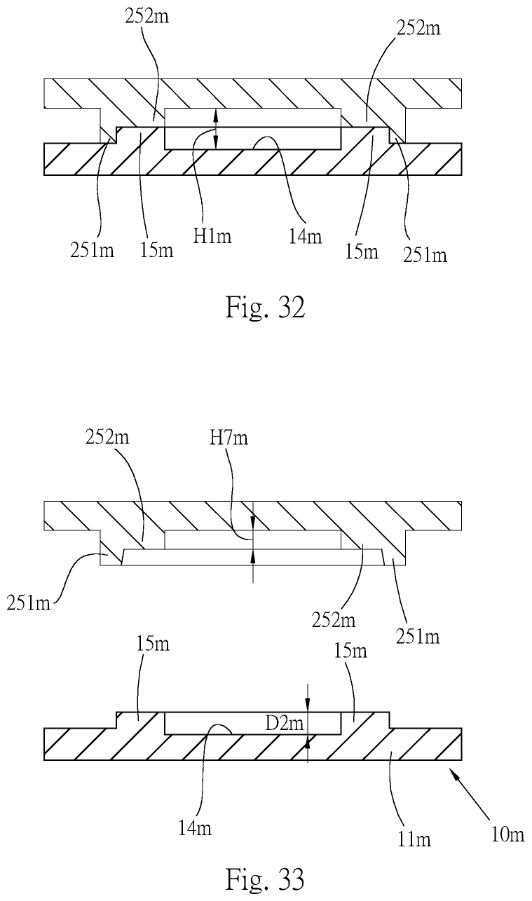

Please refer to FIG. 32 and FIG. 33 for a thirteenth embodiment of the invention with main differences different from the aforementioned twelfth embodiment lying in a first plate 10m further including a groove 14m located on a protruding portion 15m, and a predetermined groove depth D2m is recessed from a first end surface 12m toward an inner direction of a first plate body 11m. Furthermore, since a first sleeve body 251m and a second sleeve body 252m are of unequal structural design, a free end of the second sleeve body 252m abuts on the first end surface 12m, and a sum of the groove depth D2m of the groove 14m and a height H7m of the second sleeve body 252m constitutes an accommodating height H1m.

It is to be understood that the above description is only preferred embodiments of the present invention and is not used to limit the present invention, and changes in accordance with the concepts of the present invention may be made without departing from the spirit of the present invention, for example, the equivalent effects produced by various transformations, variations, modifications and applications made to the configurations or arrangements shall still fall within the scope covered by the appended claims of the present invention.

What is claimed is:

1. A container with an embedded structure comprising:
a first plate with a first plate body, a first end surface located on one side of the first plate body;
a second plate with a second plate body, a second end surface opposite to the first end surface and located on one side of the second plate body, a flexible protrusion being annularly protruded on the second end surface and abutting against the first plate to form an accommodating space between the first end surface, the second end surface, and the protrusion for a fluid to flow in the accommodating space, and the accommodating space having an accommodating height between the first end surface and the second end surface; and
a shape of a portion of a free end of the protrusion abutting the first end surface being deformed.

2. The container with the embedded structure as claimed in claim 1, wherein the first plate further includes a concave portion complementarily embedded with the protrusion, and recessed with a predetermined depth from the first end surface toward an inner direction of the first plate body.

3. The container with the embedded structure as claimed in claim 2, wherein the protrusion further includes a first convex body inserted in the concave portion, a free end of the first convex body abuts against a bottom surface of the concave portion, a height of the first convex body protruding outside of the second end surface is greater than a depth of the concave portion, and a portion of the first convex body exposed outside of the concave portion constitutes the accommodating height.

4. The container with the embedded structure as claimed in claim 2, wherein the protrusion further includes a first convex body inserted in the concave portion, and a second convex body disposed on one side of the first convex body, a height of the first convex body protruding outside of the second end surface is greater than a height of the second convex body protruding outside of the second end surface, and a stopping distance between the free end of the first convex body and a free end of the second convex body is smaller than the depth of the concave portion, so that the free end of the second convex body abuts on the first end surface, and a height of the second convex body constitutes the accommodating height.

5. The container with the embedded structure as claimed in claim 2, wherein the protrusion further includes a first convex body inserted in the concave portion, a height of the convex body protruding from the second end surface is not greater than a depth of the concave portion; the first plate further includes a groove located on the first end surface and surrounded by the concave portion, a predetermined groove depth is recessed from the first end surface toward the inner direction of the first plate body, and the groove depth of the groove constitutes the accommodating height.

6. The container with the embedded structure as claimed in claim 2, wherein the first plate further includes a groove located on the first end surface and surrounded by the concave portion, a predetermined groove depth is recessed from the first end surface toward the inner direction of the first plate body; the protrusion further includes a first convex body inserted in the concave portion, and a second convex body with a predetermined thickness disposed on one side of the first convex body, a height of the first convex body protruding outside of the second end surface is greater than a height of the second convex body protruding outside of the second end surface, and a stopping distance between the free end of the first convex body and the free end of the second convex body is smaller than the depth of the concave portion, so that the free end of the second convex body abuts on the first end surface, and a sum of the groove depth of the groove and the height of the second convex body constitutes the accommodating height.

7. The container with the embedded structure as claimed in any one of claim 3, wherein an axial sectional width of the protrusion along its own annular center is not less than an axial sectional width of the concave portion along its own annular center.

8. The container with the embedded structure as claimed in any one of claim 4, wherein an axial sectional width of the protrusion along its own annular center is not less than an axial sectional width of the concave portion along its own annular center.

9. The container with the embedded structure as claimed in any one of claim 5, wherein an axial sectional width of the protrusion along its own annular center is not less than an axial sectional width of the concave portion along its own annular center.

10. The container with the embedded structure as claimed in any one of claim 6, wherein an axial sectional width of the protrusion along its own annular center is not less than an axial sectional width of the concave portion along its own annular center.

11. The container with the embedded structure as claimed in any one of claim 1, further including a third plate;
the third plate having a third end surface, the second plate having a fourth end surface located on another side of the second plate body and opposite to the second end surface, and the third end surface of the third plate being attached with the fourth end surface of the second plate, so that the second plate being sandwiched and pressed tightly between the first plate and the third plate.

12. The container with the embedded structure as claimed in claim 11, wherein the axial sectional width of the protrusion along its own annular center is not greater than the axial sectional width of the concave portion along its own annular center.

13. The container with the embedded structure as claimed in any one of claim 2, further including a third plate;
the third plate having a third end surface, the second plate having a fourth end surface located on another side of the second plate body and opposite to the second end surface, and the third end surface of the third plate being attached with the fourth end surface of the second plate, so that the second plate being sandwiched and pressed tightly between the first plate and the third plate.

14. The container with the embedded structure as claimed in claim 13, wherein the axial sectional width of the protrusion along its own annular center is not greater than the axial sectional width of the concave portion along its own annular center.

15. The container with the embedded structure as claimed in any one of claim 3, further including a third plate;
the third plate having a third end surface, the second plate having a fourth end surface located on another side of the second plate body and opposite to the second end surface, and the third end surface of the third plate being attached with the fourth end surface of the second plate, so that the second plate being sandwiched and pressed tightly between the first plate and the third plate.

16. The container with the embedded structure as claimed in claim 15, wherein the axial sectional width of the protrusion along its own annular center is not greater than the axial sectional width of the concave portion along its own annular center.

17. The container with the embedded structure as claimed in any one of claim 4, further including a third plate;
the third plate having a third end surface, the second plate having a fourth end surface located on another side of the second plate body and opposite to the second end surface, and the third end surface of the third plate being attached with the fourth end surface of the second plate, so that the second plate being sandwiched and pressed tightly between the first plate and the third plate.

18. The container with the embedded structure as claimed in claim 17, wherein the axial sectional width of the protrusion along its own annular center is not greater than the axial sectional width of the concave portion along its own annular center.

19. The container with the embedded structure as claimed in any one of claim 5, further including a third plate;
the third plate having a third end surface, the second plate having a fourth end surface located on another side of the second plate body and opposite to the second end surface, and the third end surface of the third plate being attached with the fourth end surface of the second plate, so that the second plate being sandwiched and pressed tightly between the first plate and the third plate.

20. The container with the embedded structure as claimed in claim 19, wherein the axial sectional width of the protrusion along its own annular center is not greater than the axial sectional width of the concave portion along its own annular center.

21. The container with the embedded structure as claimed in any one of claim 6, further including a third plate;
the third plate having a third end surface, the second plate having a fourth end surface located on another side of the second plate body and opposite to the second end surface, and the third end surface of the third plate being attached with the fourth end surface of the second plate, so that the second plate being sandwiched and pressed tightly between the first plate and the third plate.

22. The container with the embedded structure as claimed in claim 21, wherein the axial sectional width of the protrusion along its own annular center is not greater than the axial sectional width of the concave portion along its own annular center.

23. A container with an embedded structure comprising:
a first plate with a first plate body, a first end surface located on one side of the first plate body; and
a second plate with a second plate body, a second end surface opposite to the first end surface and located on one side of the second plate body, a sleeve portion annularly protruded on the second end surface to sleeve on the first plate to achieve a tightly connected state between the first plate and the sleeve portion in order to form an accommodating space between the first end surface, the second end surface, and the sleeve portion for a fluid to flow in the accommodating space, and the accommodating space having an accommodating height between the first end surface and the second end surface.

24. The container with the embedded structure as claimed in claim 23, wherein the second plate is made of flexible material.

25. The container with the embedded structure as claimed in claim 24, wherein the first plate further includes a protruding portion protruding on the first end surface, an outer diameter of the protruding portion is greater than an inner diameter of the sleeve portion, when an inner side surface of the sleeve portion is sleeved on a peripheral side of the protruding portion, the second plate is deformed to change the inner diameter of the sleeve portion.

26. The container with the embedded structure as claimed in claim 25, wherein a height of the protruding portion protruding from the first end surface is smaller than a height of the sleeve portion protruding from the second end surface.

27. The container with the embedded structure as claimed in claim 25, wherein the first plate further includes a groove located on the protruding portion, a predetermined groove depth is recessed from the first end surface toward the inner direction of the first plate body, and a groove depth of the groove constitutes the accommodating height.

28. The container with the embedded structure as claimed in claim 25, wherein the sleeve portion further includes a first sleeve body sleeved on the peripheral side of the protruding portion, and a second sleeve body provided on one side of the first sleeve body, a height of the first sleeve body protruding outside of the second end surface is greater than a height of the second sleeve body protruding outside of the second end surface, and a stopping distance between a free end of the first sleeve body and a free end of the second sleeve body is smaller than a height of the protruding portion, so that the free end of the second sleeve body abuts on the protruding portion, and a height of the second sleeve body constitutes the accommodating height.

29. The container with the embedded structure as claimed in claim 25, wherein the first plate further includes a groove located on the protruding portion, a predetermined groove depth is recessed from the first end surface toward the inner direction of the first plate body; the sleeve portion further includes a first sleeve body sleeved on the peripheral side of the protruding portion, and a second sleeve body provided on one side of the first sleeve body, a height of the first sleeve body protruding outside of the second end surface is greater than a height of the second sleeve body protruding outside of the second end surface, and a stopping distance between the free end of the first sleeve body and the free end of the second sleeve body is smaller than a height of the protruding portion, so that the free end of the second sleeve body abuts on the protruding portion, and a sum of the groove depth of the groove and the height of the second sleeve body constitutes the accommodating height.
